(19) United States
(12) Patent Application Publication
(10) Pub. No.: US 2025/0197792 A1
(43) Pub. Date: Jun. 19, 2025

(54) INTEGRATION OF ADSORPTION DEVICE AND GAS FERMENTATION

(71) Applicant: LanzaTech, Inc., Skokie, IL (US)

(72) Inventors: Darren Earl Youngs, Chicago, IL (US); Robert John Conrado, Washington, DC (US)

(21) Appl. No.: 18/544,828

(22) Filed: Dec. 19, 2023

(51) Int. Cl. C12M 1/00; B01D 53/04; B01D 53/047; B01D 53/14; C01B 3/56; C10K 1/00

(52) U.S. Cl. CPC C12M 29/26; B01D 53/0462; B01D 53/047; B01D 53/1487; C01B 3/56; C10K 1/005; C12M 21/12; C12M 47/18; B01D 2256/16; B01D 2256/20; B01D 2257/504; B01D 2258/02; B01D 2258/05; B01D 2259/40001; B01D 2259/40007; B01D 2259/40013; B01D 2259/40028; B01D 2259/40054; B01D 2259/4009; C01B 2203/80

(57) ABSTRACT

An integrated temperature swing adsorption (TSA) process and gas fermentation process and device is disclosed. A heated tail gas stream from the gas fermentation process is used to heat and regenerate adsorbent in the TSA device. A portion of treated feedstock from the TSA device is used to cool the regenerated adsorbent. Integration of a tail gas stream from the gas fermentation zone used for regeneration of absorbent in the TSA eliminates the need for an inert gas regenerant and using TSA treated gas feedstock for cooling regenerated adsorbent allows for maximum recovery and use of available gas feedstock. Alternatively, when a pressure swing adsorption (PSA) process is also employed, a purge stream from the PSA may be used as regenerant in the TSA process.

### FIELD

This disclosure relates to a method and device for the integration of temperature swing adsorption and gas fermentation. The substrate for gas fermentation, is passed through the adsorption device for removal of impurities to ensure safe gas fermentation.

### BACKGROUND

Carbon dioxide (CO₂) accounts for about 76% of global greenhouse gas emissions from human activities, with methane (16%), nitrous oxide (6%), and fluorinated gases (2%) accounting for the balance (United States Environmental Protection Agency). The majority of CO₂ comes from the burning fossil fuels to produce energy, although industrial and forestry practices also emit CO₂ into the atmosphere. Reduction of greenhouse gas emissions, particularly CO₂, is critical to halt the progression of global warming and the accompanying shifts in climate and weather.

It has long been recognized that catalytic processes, such as the Fischer-Tropsch process, may be used to convert gases containing carbon dioxide (CO₂), carbon monoxide (CO), and/or hydrogen (H₂), such as industrial waste gas or syngas or mixtures thereof into a variety of chemicals such as ethanol, acetone, and isopropanol. Syngas can also be converted to various chemicals by the Monsanto process by converting to methanol as a first step. Both Fischer-Tropsch and Methanol synthesis units are optimized at very high capacities. They require well defined feed gas compositions and syngas feed with low impurities to avoid poisoning the catalysts. Fischer-Tropsch process requires complex and costly purification equipment to generate high purity industrial chemicals. Recently, gas fermentation has emerged as an alternative platform for the biological fixation of such gases. C1-fixing microorganisms have been demonstrated to convert gases containing CO₂. CO, and/or H₂ such as industrial waste gas or syngas or mixtures thereof into products such as ethanol and 2,3-butanediol.

Gas fermentation has emerged as an alternative platform for biological capture and transformation of carbon in gases containing single carbon atom compounds such as carbon monoxide (CO), carbon dioxide (CO₂), and/or methane (CH₄). In particular, C1-fixing microorganisms act as biocatalysts to convert a C1-carbon gaseous substrate into valuable fermentation products such as ethanol or other C1-C4 alcohols.

Temperature swing adsorption (TSA) may be used to remove contaminates from a gaseous feedstock. When one TSA bed becomes ineffective at removing the desired impurities to the desired concentration due to the adsorbent nearing adsorption capacity limits, termed “saturation”, the adsorbent may be regenerated. The adsorbent bed is comprised of at least one adsorbent selected to remove the target impurity. Additional adsorbents can be added to the adsorbent bed to target multiple impurities. Adsorbent regeneration is achieved by passing a heated gas through the adsorbent bed to transfer heat from the regeneration gas to the adsorbent. As the adsorbent temperature increases, the impurities are desorbed from the adsorbent due to the saturation capacity of the adsorbent decreases with increasing temperature. The regeneration gas flow rate is sufficient to ensure adequate mixing and to ensure the regeneration gas is not saturated with the desorbed impurities. Typically, an inert gas such as nitrogen is used as the regeneration gas. However, production of the inert gas involves expensive equipment thus adding to overall costs. Additionally, the impurity-laden inert gas must be further treated before release to the atmosphere or repurposed. A typical treatment method is thermal oxidation at high temperatures. Given the inert nature of the regeneration gas, significant energy is required to heat the impurity laden inert gas up to the required thermal oxidation temperature. Alternatively, a portion of the untreated gaseous feedstock can be heated and used as a regeneration gas. However, this reduces the quantity of gaseous substrate available for fermentation. Accordingly, a need exists for a method and device for regenerating a saturated adsorption bed other than using an inert gas for regeneration while also preserving gaseous feedstock for use in the process and not lost to regeneration.

### BRIEF SUMMARY

In one aspect of the disclosure, a process for an integrated TSA process and gas fermentation process comprises (a) providing a TSA process comprising at least a first adsorption bed comprising adsorbent and a second adsorption bed comprising spent or saturated adsorbent and a gas fermentation process comprising a bioreactor comprising at least one C1-fixing microorganism in a nutrient solution; (b) passing a gaseous feedstock comprising CO, CO₂, H₂, CH₄, or any combination thereof, and at least one contaminant (or impurity) to the first adsorption bed operating at an adsorption temperature, adsorbing the contaminant on the adsorbent, and generating a treated gaseous feedstock depleted in the contaminant; (c) passing at least a portion of the treated gaseous feedstock to the gas fermentation process to generate a gas fermentation product stream comprising a gas fermentation product and a tail gas stream; (d) first passing at least a portion of the tail gas stream to a heater to produce a heated tail gas stream from the gas fermentation process at a regeneration temperature to the second adsorption bed and through the spent adsorbent to desorb the adsorbed contaminant and provide a heated regenerated adsorbent, wherein the regeneration temperature is greater than the adsorption temperature; (c) then passing at least a portion of the treated gaseous feedstock from the first adsorption bed to the second adsorption bed and through the regenerated adsorbent to cool the heated regenerated adsorbent to the adsorption temperature and provide an effluent stream from the regenerated bed; and (f) combining the effluent stream from the second adsorption bed with the treated gaseous feedstock from the first adsorption bed or passing the effluent stream of the treated gaseous feedstock to the gas fermentation process, or both. In general, tail gases are the gases and vapours which may be released into the atmosphere from a process after reactions and treatments have taken place. The process steps (a) to (f) are repeated periodically. In another embodiment, the process further comprises a third adsorption bed comprising regenerated adsorbent in standby mode. In another embodiment, the process further comprises more than three adsorption beds comprising regenerated adsorbent in standby mode. In yet another embodiment the process further comprises passing the heated tail gas stream from the gas fermentation process through a scrubber prior to passing to the first adsorption bed to remove and recover gas fermentation product from the tail gas stream.

The process further comprises adjusting (a) the operating pressure of the first adsorption bed to correspond with the pressure of the gaseous feedstock, and or (b) the second adsorption bed to correspond with the pressure of the heated tail gas stream from the gas fermentation process. The gas fermentation process may operate at a lower pressure relative to the adsorption pressure or at a higher pressure relative to the adsorption pressure.

In another aspect of the disclosure, a process for an integrated TSA process, pressure swing adsorption (PSA) process, and gas fermentation process comprises (a) providing a TSA process comprising at least a first TSA adsorption bed comprising TSA adsorbent and a second TSA adsorption bed comprising TSA spent adsorbent, a pressure swing adsorption process comprising at least a first PSA adsorption bed comprising PSA adsorbent and a second PSA adsorption bed comprising PSA spent adsorbent, and a gas fermentation process comprising a bioreactor comprising at least one C1-fixing microorganism in a nutrient solution; (b) passing a gaseous feedstock comprising CO, CO₂, H₂, CH₄, or any combination thereof, and at least one contaminant to the first TSA adsorption bed operating at an adsorption temperature, adsorbing the contaminant on the TSA adsorbent, and generating a TSA treated gaseous feedstock depleted in the contaminant; (c) passing at least a portion of the treated gaseous feedstock to the first PSA adsorption bed operating at an adsorption pressure, adsorbing CO₂ on the PSA adsorbent, and generating a PSA treated gaseous feedstock depleted in CO₂; (d) passing the PSA treated gaseous feedstock to a gas fermentation process to generate a gas fermentation product stream comprising a gas fermentation product; (c) regenerating the first PSA bed by reducing the operating pressure below the adsorption pressure resulting in a PSA purge gas stream enriched in CO₂; (f) then passing at least a portion of the PSA purge gas stream from the second PSA adsorption bed to the second TSA adsorption bed at a desorption temperature to desorb the adsorbed contaminant and provide a TSA purge gas comprising the contaminant and CO₂. In another embodiment, the gaseous feedstock comprises methane, hydrogen sulfide and or other light hydrocarbons and the PSA bed absorbs the methane or other light hydrocarbons (C₁-C₁₃) or hydrogen sulfide. In one embodiment, a compressor is used to increase the pressure of the PSA purge gas to sufficiently high pressure to overcome the pressure drop of the second TSA bed and associated equipment.

In another aspect of the disclosure, a process for an integrated temperature swing adsorption process, pressure swing adsorption process, and gas fermentation process comprises providing a temperature swing adsorption process comprising at least a first TSA adsorption bed comprising TSA adsorbent and a second TSA adsorption bed comprising TSA spent adsorbent, a pressure swing adsorption process comprising at least a first PSA adsorption bed comprising PSA adsorbent and a second PSA adsorption bed comprising PSA spent adsorbent, and a gas fermentation process comprising a bioreactor comprising at least one C1-fixing microorganism in a nutrient solution; passing a gaseous feedstock comprising CO, CO₂, H₂, CH₄, or any combination thereof, and at least one contaminant to the first TSA adsorption bed operating at an adsorption temperature, adsorbing the contaminant on the TSA adsorbent, and generating a TSA treated gaseous feedstock depleted in the contaminant; passing at least a portion of the treated gaseous feedstock to a gas fermentation process to generate a fermentation tail gas and a gas fermentation product stream comprising a gas fermentation product; passing at least a portion of the fermentation tail gas to the first PSA adsorption bed operating at an adsorption pressure, adsorbing CO₂ on the PSA adsorbent, and generating a PSA treated fermentation tail gas depleted in CO₂ and PSA purge gas; passing at least a portion of the PSA purge gas from the first PSA adsorption bed to the second TSA adsorption bed at a desorption temperature to desorb the adsorbed contaminant and provide a TSA purge gas comprising the contaminant and CO₂.

In another aspect of the disclosure, an integrated TSA and gas fermentation device comprises (i) a gaseous feedstock source in fluid communication with a first adsorption bed comprising an adsorbent, the first adsorption bed further comprising a treated gaseous feedstock outlet; (ii) a second adsorption bed comprising a spent adsorbent and having an effluent outlet wherein the treated gaseous feedstock outlet of the first adsorption bed is in further fluid communication with the second adsorption bed; and (iii) a bioreactor of a gas fermentation device in fluid communication with the treated gaseous feedstock outlet of the first adsorption bed and the effluent outlet of the second adsorption bed, the bioreactor comprising a tail gas stream outlet and a product stream outlet, the tail gas stream outlet in fluid communication with the second adsorption bed. The device, further comprising a third adsorption bed comprising regenerated adsorbent in standby mode, the third adsorption bed is in fluid communication with the bioreactor. In one embodiment, a scrubber is in fluid communication with the tail gas stream outlet of the bioreactor, the scrubber is in further fluid communication with the second adsorption bed. In yet another embodiment, the device further comprises a regeneration heater in fluid communication with the tail gas stream outlet of the bioreactor.

In yet another aspect of the disclosure, an integrated TSA, pressure swing adsorption and gas fermentation device comprises: (i) a gaseous feedstock source unit in fluid communication with a first TSA adsorption bed comprising an adsorbent, the first TSA adsorption bed further comprising a treated gaseous feedstock outlet; (ii) a first PSA adsorption bed comprising PSA adsorbent, the first PSA bed in fluid communication with the treated gaseous feedstock outlet of the first TSA adsorption bed wherein the first PSA adsorption bed is in further fluid communication with the bioreactor; and (iii) a second PSA adsorption bed comprising PSA spent adsorbent in fluid communication with the treated gaseous feedstock outlet of the first TSA adsorption bed wherein the second PSA adsorption bed is in further fluid communication with a second TSA adsorption bed comprising TSA spent adsorbent.

### BRIEF DESCRIPTION OF THE DRAWINGS

The Figures have been simplified by the deletion of a large number of apparatuses customarily employed in a process of this nature, such as vessel internals, temperature and pressure control devices, flow control valves, compressors and the like. which are not specifically required to illustrate the performance of the invention. Furthermore, the illustration of the process of this invention in the embodiment of a specific drawing is not intended to limit the disclosure to specific embodiments. Some embodiments may be described by reference to the process configuration shown in the figure, which relates to both apparatus and processes to carry out the disclosure. Any reference to a process step includes reference to an apparatus unit or equipment that is suitable to carry out the step, and vice-versa.

FIGS. 1A and 1B are schematic flow diagrams showing three TSA beds according to an embodiment of the disclosure.

FIGS. 2A and 2B are schematic flow diagrams showing two TSA beds and two PSA beds (PSA) according to an embodiment of the disclosure.

### DETAILED DESCRIPTION

In an integrated TSA and gas fermentation device, near-complete or complete recovery of treated gaseous feedstock and the elimination of the need for inert regeneration gas are benefits realized by the method and device of the disclosure. For case of understanding the disclosure is explained in terms of a TSA swing bed device of two or more TSA beds. A first TSA bed is online and in operation while a second TSA bed is being regenerated or has been regenerated and is ready for service. At a point in time where the first TSA bed approaches adsorption capacity or becomes saturated, the first TSA bed is “swung” out of service for regeneration, and the second TSA bed is “swung” into on-line service. The process continues with at least one TSA bed in service while another is being regenerated or is in a ready state for service. In this disclosure, a saturated TSA bed is regenerated using a “heated” tail gas stream from a gas fermentation process and then cooled using treated gaseous feedstock from another TSA bed of the TSA swing bed device. In another aspect, a stream from a PSA bed is used to regenerate a TSA bed. Accordingly, regeneration of TSA beds does not require inert regeneration gas as is common practice, thus eliminating the expense to acquire or generate inert regeneration gas. Further, nearly complete recovery of treated gaseous feedstock, even when used in the regeneration process, is achieved.

The gas fermentation zone of the device comprises at least one biorcactor comprising at least one C1-fixing microorganism. The term “bioreactor” includes a fermentation device consisting of one or more vessels and/or towers or piping arrangements which includes, continuous stirred tank reactor, immobilized cell reactor, trickle bed reactor, bubble column, gas lift fermenter, static mixer, circulated loop reactor, membrane reactor, such as a hollow fibre membrane bioreactor, or other vessel or other device suitable for a gas-liquid contact. The bioreactor may also comprise a device of multiple reactors (stages) either in parallel or in series. For example, the bioreactor may comprise a first growth reactor which cultures a microorganism and a second fermentation reactor to which output from the growth reactor may be fed and produces most of the fermentation products. In some embodiments, multiple bioreactors in a bioreactor device are placed on top of one-another to form a stack. A stack of bioreactors improve throughput of the bioreactor device without significantly increasing demand for land area. In some embodiments, the bioreactors include down-flow or up-flow bioreactors having mechanisms to form smaller bubbles of substrate in the liquid medium and therefore substantially improve rate of the gas-liquid mass transfer without increasing energy consumption.

A “C1-fixing microorganism” is a microorganism that produces one or more products from a C1-carbon source or substrate. The “C1-carbon source or substrate” refers to a one carbon-molecule that serves as a partial or sole carbon source for the microorganism. For example, the C1-carbon source or substrate may comprise one or more of CO, CO₂, CH₄, CH₃OH, or CH₂O₂. In an embodiment, the C1-carbon source or substrate comprises one or both of CO and CO₂. In addition to the C1-compounds, the C1-containing gaseous substrate may further comprise other non-carbon components, such as H₂, N₂, and/or electrons.

The terms “feedstock” when used in the context of the stream flowing into a gas fermentation bioreactor (i.e., gas fermenter) or “gas fermentation feedstock” should be understood to encompass any material (solid, liquid, or gas) or stream that can provide a substrate to a gas fermenter or bioreactor either directly or after processing of the feedstock.

Generally, the gas fermentation feedstock includes, but is not limited to, industrial waste gas, a syngas, a biogas, a landfill gas, a direct air capture gas, or any combination thereof. A portion of the substrate and/or C1-carbon source may be a gas obtained as a by-product of an industrial process or from another source, such as combustion engine exhaust fumes, biogas, landfill gas, direct air capture, flaring, or from electrolysis. However, a portion of the substrate and/or C1-carbon source is syngas generated by pyrolysis, torrefaction, or gasification. In other words, carbon in solid or liquid materials may be recycled by pyrolysis, torrefaction, or gasification to generate syngas which is used as the substrate and/or C1-carbon source in gas fermentation. A portion of the substrate and/or C1-carbon source may be natural gas, carbon dioxide from conventional and unconventional gas production, and or a gas comprising methane. Gas fermentation processes are flexible and any of these substrates and/or C1-carbon sources may be employed.

In certain embodiments, the industrial process source of an optional portion of the substrate and/or C1 carbon source is selected from ferrous metal products manufacturing, such as a steel manufacturing, non-ferrous products manufacturing, petroleum refining, electric power production, carbon black production, paper and pulp manufacturing, ammonia production, methanol production, coke manufacturing, petrochemical production, carbohydrate fermentation, cement making, aerobic digestion, anaerobic digestion, catalytic processes, natural gas extraction, cellulosic fermentation, oil extraction, industrial processing of geological reservoirs, processing fossil resources such as natural gas coal and oil, landfill operations, silicon carbide production, or any combination thereof. Examples of specific processing steps within an industrial process include catalyst regeneration, fluid catalyst cracking, and catalyst regeneration. Air separation and direct air capture are other suitable industrial processes. Specific examples in steel and ferroalloy manufacturing include blast furnace gas, basic oxygen furnace gas, coke oven gas, direct reduction of iron furnace top-gas, and residual gas from smelting iron. Other general examples include flue gas from fired boilers and fired heaters, such as natural gas, oil, or coal fired boilers or heaters, and gas turbine exhaust. Another example is the flaring of compounds such as at oil and gas production sites. In these embodiments, the substrate and/or C1-carbon source may be captured from the industrial process before it is emitted into the atmosphere, using any known method.

The substrate and/or C1-carbon source is synthesis gas known as syngas, which may be obtained from reforming, partial oxidation, plasma, or gasification processes. Examples of gasification processes include gasification of coal, gasification of refinery residues, gasification of petroleum coke, gasification of biomass, gasification of lignocellulosic material, gasification of wood, gasification of black liquor, gasification of municipal solid waste, gasification of municipal liquid waste, gasification of industrial solid waste, gasification of industrial liquid waste, gasification of refuse derived fuel, gasification of sewerage, gasification of sewerage sludge, gasification of sludge from wastewater treatment, gasification of landfill gas, gasification of biogas such as when biogas is added to enhance gasification of another material, gasification of rubber containing material, including portions of tires and whole tires. Examples of reforming processes include, steam methane reforming, steam naphtha reforming, reforming of natural gas, reforming of biogas, reforming of landfill gas, reforming of coke oven gas, reforming of pyrolysis off-gas, reforming of ethylene production off-gas, naphtha reforming, and dry methane reforming. Examples of partial oxidation processes include thermal and catalytic partial oxidation processes, catalytic partial oxidation of natural gas, partial oxidation of hydrocarbons, partial oxidation of biogas, partial oxidation of landfill gas, or partial oxidation of pyrolysis off-gas. Examples of municipal solid waste include tires, plastics, refuse derived fuel, and fibers such as in shoes, apparel, and textiles. Municipal solid waste may be simply landfill-type waste and may be sorted or unsorted. Tires including end of file tires may be a feedstock. Examples of biomass may include lignocellulosic material and microbial biomass. Lignocellulosic material may include agriculture by-products, forest by-products, and some industrial by-products. Whole tires may be processed by pyrolysis to form syngas.

Biomass may be created as by-products of “nature-based solutions” (NBS) and thus natured-based solutions may provide feedstock to the gas fermentation process. Nature-based solutions is articulated by the European Commission as solutions inspired and supported by nature, which are cost-effective, simultaneously provide environmental, social, and economic benefits and help build resilience. Such solutions bring more, and more diverse, nature and natural features and processes into cities, landscapes, and seascapes, through locally adapted, resource-efficient, and systemic interventions. Nature-based solutions must benefit biodiversity and support the delivery of a range of ecosystem services. Through the use of NBS healthy, resilient, and diverse ecosystems (whether natural, managed, or newly created) can provide solutions for the benefit of both societies and overall biodiversity. Examples of nature-based solutions include natural climate solutions (conservation, restoration and improved land management that increase carbon storage or avoid greenhouse gas emissions in landscapes and wetlands across the globe), halting biodiversity loss, socio-economic impact efforts, habitat restoration, and health and wellness efforts with respect to air and water. Biomass produced through nature-based solutions may be used as feedstock to gas fermentation processes.

A portion of the substrate and/or C1-carbon source may be a gas stream comprising methane. Such a methane containing gas may be obtained from: fossil methane emissions such as during fracking, wastewater treatment, livestock, agriculture, and municipal solid waste landfills. It is also envisioned that the methane may be burned to produce electricity or heat and the C1 by-products may be used as the substrate or carbon source. The substrate and/or C1-carbon source may be a gas stream comprising natural gas.

The syngas substrate generally contains a major proportion of CO, such as at least about 15% to about 75% CO by volume, from about 20% to about 70% CO by volume, from about 20% to about 65% CO by volume, from about 20% to about 60% CO by volume, and from about 20% to about 55% CO by volume. In some embodiments, the syngas substrate comprises about 25% CO, or about 30% CO, or about 35% CO, or about 40% CO, or about 45% CO, or about 50% CO, or about 55% CO, or about 60% CO by volume. In other embodiments, the syngas substrate generally contains a major proportion of CO₂, such as at least about 15% to about 75% CO₂ by volume, from about 20% to about 70% CO₂ by volume, from about 20% to about 65% CO₂ by volume, from about 20% to about 60% CO₂by volume, and from about 20% to about 55% CO₂ by volume. In some embodiments, the syngas substrate comprises about 25% CO₂, or about 30% CO₂, or about 35% CO₂, or about 40% CO₂, or about 45% CO₂, or about 50% CO₂, or about 55% CO, or about 60% CO₂ by volume.

The C1-fixing microorganisms may be selected from Clostridium, Moorella, Pyrococcus, Eubacterium, Desulfobacterium, Carboxydothermus, Acetogenium, Acetobacterium, Acetoanaerobium, Butyribaceterium, Peptostreptococcus and mixtures thereof. The microorganisms in the bioreactor may be a modified microorganism derived from a naturally occurring or parental microorganism. A “parental microorganism” is a microorganism from which another microorganism can be derived. Both the parental and derived microorganism are suitable microorganisms. The parental microorganism may be a naturally-occurring microorganism (i.e., a wild-type microorganism) or a microorganism previously modified (i.e., an optimized, a mutant or recombinant microorganism). Suitable microorganisms may be modified to express or overexpress one or more enzymes that were not expressed or overexpressed in the parental microorganism. Similarly, microorganisms of the disclosure may be modified to contain one or more genes that were not contained in the parental microorganism. The microorganisms may also be modified to not express or to express lower amounts of one or more enzymes that were expressed in the parental microorganism. In accordance with one embodiment, the microorganism is selected from or derived from Acetobacterium woodii, Alkalibaculum bacchii, Blautia producta, Butyribacterium methylotrophicum, Clostridium aceticum, Clostridium autoethanogenum, Clostridium carboxidivorans, Clostridium coskatii, Clostridium drakei, Clostridium formicoaceticum, Clostridium ljungdahlii, Clostridium magnum, Clostridium ragsdalei, Clostridium scatologenes, Eubacterium limosum, Moorella thermautotrophica, Moorella thermoacetica, Oxobacter pfennigii, Sporomusa ovata, Sporomusa silvacetica, Sporomusa sphaeroides, Cupriavidus necator, Thermoanaerobacter kivui, or any combination thereof. In one embodiment, the microorganism is selected from or derived from Clostridium autoethanogenum, Clostridium ljungdahlii, or Clostridium ragsdalei. In one embodiment, the parental microorganism is Clostridium autoethanogenum which was deposited on Jun. 7, 2010, with Deutsche Sammlung von Mikroorganismen und Zellkulturen GmbH (DSMZ) located at Inhoffenstraβe 7B, D-38124 Braunschweig, under the terms of the Budapest Treaty and accorded accession number DSM23693. This strain is described in International Patent Application No. PCT/NZ2011/000144, which is published as WO 2012/015317.

The microorganisms of the disclosure may be cultured with the gaseous substrate to produce one or more products. For instance, the microorganism may produce or may be engineered to produce ethanol (WO 2007/117157, U.S. Pat. No. 7,972,824), acetate (WO 2007/117157, U.S. Pat. No. 7,972,824), 1-butanol (WO 2008/115080, U.S. Pat. No. 8,293,509, WO 2012/053905, U.S. Pat. No. 9,359,611 and WO 2017/066498, U.S. Pat. No. 9,738,875), butyrate (WO 2008/115080, U.S. Pat. No. 8,293,509), 2,3-butanediol (WO 2009/151342, U.S. Pat. No. 8,658,408 and WO 2016/094334, U.S. Pat. No. 10,590,406), lactate (WO 2011/112103, U.S. Pat. No. 8,900,836), butene (WO 2012/024522, US2012/045807), butadiene (WO 2012/024522, US 2012/045807), methyl ethyl ketone (2-butanone) (WO 2012/024522, US 2012/045807 and WO 2013/185123, U.S. Pat. No. 9,890,384), ethanol which is then converted to ethylene (WO 2012/026833, US 2013/157,322), acetone (WO 2012/115527, U.S. Pat. No. 9,410,130), isopropanol (WO 2012/115527 U.S. Pat. No. 9,410,130), lipids (WO 2013/036147 U.S. Pat. No. 9,068,202), 3-hydroxypropionate (3-HP) (WO 2013/180581, U.S. Pat. No. 9,994,878), terpenes, including isoprene (WO 2013/180584, U.S. Pat. No. 10,913,958), fatty acids (WO 2013/191567 U.S. Pat. No. 9,347,076), 2-butanol (WO 2013/185123 U.S. Pat. No. 9,890,384), 1,2-propanediol (WO 2014/036152, U.S. Pat. No. 9,284,564), 1 propanol (WO 2014/0369152, U.S. Pat. No. 9,284,564), 1 hexanol (WO 2017/066498, U.S. Pat. No. 9,738,875), 1 octanol (WO 2017/066498, U.S. Pat. No. 9,738,875), chorismate-derived products (WO 2016/191625, U.S. Pat. No. 10,174,303), 3 hydroxybutyrate (WO 2017/066498, U.S. Pat. No. 9,738,875), 1,3-butanediol (WO 2017/066498, U.S. Pat. No. 9,738,875), 2-hydroxyisobutyrate or 2-hydroxyisobutyric acid (WO 2017/066498, U.S. Pat. No. 9,738,875), isobutylene (WO 2017/066498, U.S. Pat. No. 9,738,875), adipic acid (WO 2017/066498, U.S. Pat. No. 9,738,875), 1,3 hexanediol (WO 2017/066498, U.S. Pat. No. 9,738,875), 3-methyl-2-butanol (WO 2017/066498, U.S. Pat. No. 9,738,875), 2-buten-1-ol (WO 2017/066498, U.S. Pat. No. 9,738,875), isovalerate (WO 2017/066498, U.S. Pat. No. 9,738,875), isoamyl alcohol (WO 2017/066498, U.S. Pat. No. 9,738,875), and/or monocthylene glycol (WO 2019/126400, U.S. Pat. No. 11,555,209) in addition to 2-phenylethanol (WO 2021/188190, US 2021/0292732) and ethylenc.

In certain embodiments, microbial biomass itself may be considered a product. These products may be further converted to produce at least one component of diesel, jet fuel, sustainable aviation fuel (SAF) and/or gasoline. In certain embodiments, ethylene may be catalytically converted into another product, article, or any combination thereof. Additionally, the microbial biomass may be further processed to produce a single cell protein (SCP) by any method or combination of methods known in the art. The fermentation broth effluent from the bioreactor comprises the product(s) generated and the medium present in the bioreactor.

The bioreactor(s) of the gas fermentation process are operated suitable conditions to produce a product. Specific reaction conditions will depend partly on the particular microorganism used and the target product. However, in general, it is preferable to operate the fermentation at a pressure higher than atmospheric pressure and temperatures suitable for the specific microorganism employed.

Target products may be separated or purified from the fermentation broth effluent using any method or combination of methods known in the art, including, for example, fractional distillation, evaporation, pervaporation, gas stripping, phase separation, and extractive fermentation, including, for example, liquid-liquid extraction. In certain embodiments, target products are recovered from the fermentation broth by continuously removing a portion of the broth from the bioreactor, separating microbial biomass from the broth, such as by filtration including membrane separation, and recovering one or more target products from the permeate. Alcohols and/or acetone may be recovered, for example, by distillation. Acids may be recovered, for example, by adsorption on activated charcoal. Separated microbial biomass, such as that in a retentate, may be returned to the bioreactor. A portion of microbial cell-free permeate remaining after target products have been removed may be returned to the bioreactor to recycle medium. Additional nutrients may be added to the recycled cell-free permeate to replenish the medium before being recycled to the bioreactor. Product and microbial biomass may be recovered in one operation using vacuum distillation.

Substrate to the gas fermentation process is typically treated to remove fermentation inhibiting components before being introduced to the bioreactor. A TSA device may be employed to adsorb selective contaminants in a gas feedstock onto an adsorbent at an adsorption temperature. Upon saturation of the adsorbent, the adsorbed contaminants are desorbed from the adsorbent at a desorption temperature and the TSA bed is thereby regenerated and may be used again. Suitable adsorbents for use in the TSA beds include, but are not limited to, zeolitic molecular sieves, activated carbon, silica gel, activated aluminas, or any combination thereof. Typical fermentation inhibitors removed by adsorption onto the TSA beds may include, but are not limited to hydrocarbon, an oxygenate, a sulfur compound, a nitrogen compound, or any combination thereof. If present, lighter tars with lower boiling points, such as benzene, toluene, ethylbenzene, and xylene (BTEX), may also be removed from feedstocks.

Referring to a single bed of a TSA device, the steps of a TSA process as applied to that single bed include:
1) an adsorption step at an adsorption pressure and an adsorption temperature;
2) an optional depressurization step to lower the pressure to a regeneration pressure which may be lower than adsorption pressure;
3) a desorption step at a desorption temperature which is higher than the adsorption temperature;
4) a cooling step to return the TSA bed from the desorption temperature to the adsorption temperature; and
5) a pressurization step to return the TSA bed from the regeneration pressure to the adsorption pressure.

The adsorption step lasts until the adsorbent no longer has sufficient available adsorption capacity to remove target components to the target concentration. The desperation step lasts until sufficient available adsorption capacity to remove target components has been restored to the TSA bed. The adsorption step may be considered an adsorption mode, while the depressurization, desorption, cooling, and pressurization steps maybe considered a regeneration mode.

In swing bed operation, while a first TSA bed is in the adsorption step and in service, a second TSA bed may be undergoing the depressurization, desorption, cooling, and pressurization steps. Yet a third TSA bed may be in a stand-by mode, fully regenerated and ready to be placed in service. Any number of TSA beds may be in active service, in regeneration, or in standby mode. For simplicity, the disclosure is focused on a two-bed TSA process. However, it is understood that a three-bed TSA process or even a four-bed TSA process may be beneficial. Furthermore, regeneration of TSA beds may occur in a flow direction that is co-current or counter current to the flow direction of the adsorption mode.

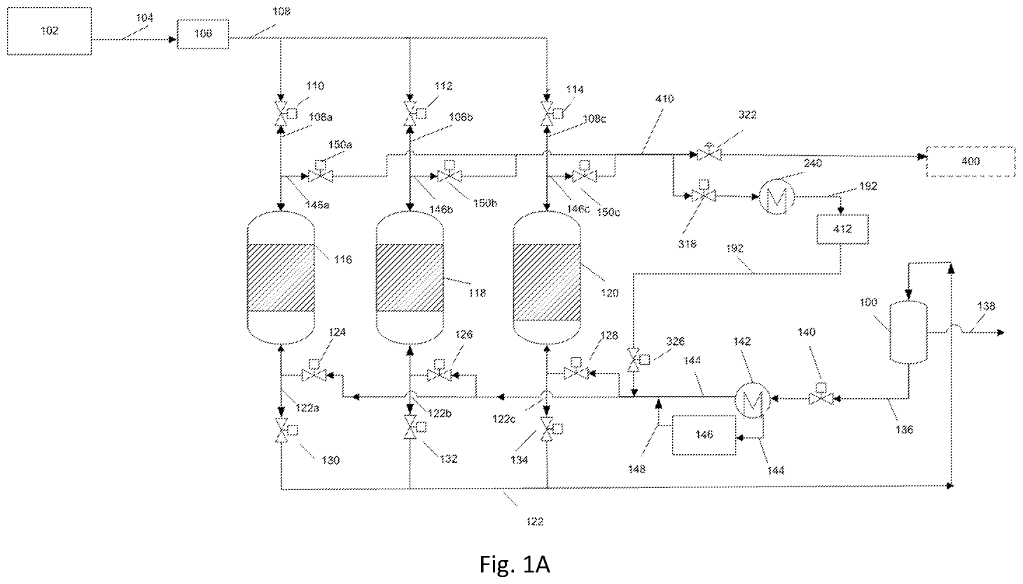

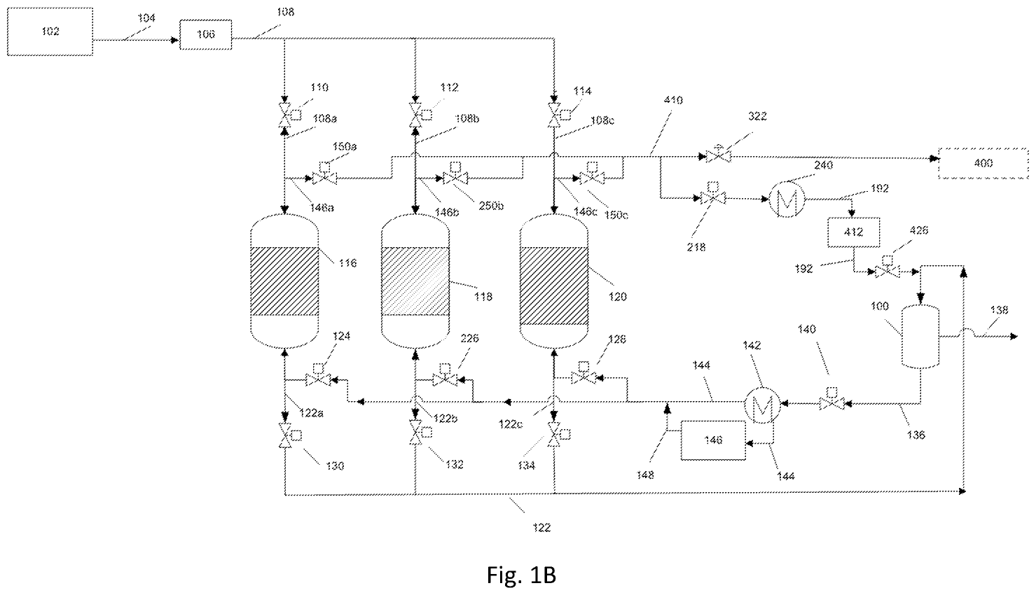

Referring to FIGS. 1A and 1B, a gas fermentation zone comprises at least a bioreactor 100 which converts treated gaseous feedstock 122 to produce a least one target product stream 138 comprising at least one product and a tail gas stream, also called a tail gas stream 136 comprising at least CO₂. Treated gaseous feedstock 122 is from a feedstock source 102 and optionally compressor 106 with subsequent treatment using a TSA process comprising adsorbent beds 116, 118, and 120. The TSA process is described with the regeneration mode of adsorbent beds occurring in a fluid flow direction that is counter-current to the fluid flow in the adsorption mode. It is envisioned that in another embodiment the regeneration mode of the adsorbent beds may occur in a co-current fluid flow direction.

Feedstock source 102 provides gaseous feedstock 104. Suitable feedstock sources are described above. Gaseous feedstock 104 is compressed in compressor 106 to generate compressed gaseous feedstock 108. Valves 110, 112, and 114 control fluid flow of compressed gaseous feedstock 108, shown as 108a, 108b, and 108c, to each of the respective adsorbent beds 116, 118, and 120. As depicted in FIGS. 1A and 1B, first adsorbent bed 116 comprises active adsorbent and is in adsorption mode while second adsorbent bed 118 comprises spent adsorbent, which has reduced adsorption capacity as compared to fresh or regenerated adsorbent. In some embodiments the spent adsorbent has reached capacity and cannot adsorb additional contaminant. Second adsorbent bed 118 as shown in FIGS. 1A and 1B is in regeneration mode. Optional third adsorbent bed 120 has been regenerated and is ready for on-line service (online service of third adsorbent bed 120 is not shown).

In an embodiment, first adsorbent bed 116, when in adsorption mode, is operated at an adsorption temperature ranging from about 40° C. to about 60° C. In other embodiments, the adsorption temperature may be, for example, from about 40° C. to about 50° C., or from about 50° C. to about 60° C. While first adsorption bed 116 is in adsorption mode, valve 110 and valve 130 are open allowing gaseous feedstock 108a to pass through first adsorbent bed 116 for treatment by adsorption of at least one target contaminant and generate treated gaseous feedstock 122a which is depleted in at least one target contaminant. Treated gaseous feedstock is largely passed to the bioreactor 100 in the gas fermentation zone as stream 122. In order to direct gaseous feedstock 108a to the first adsorbent bed 116, valves 112 and 114 remain closed. Additionally, valve 150a and valve 124 are closed during adsorption.

Second adsorbent bed 118 has diminished adsorption capacity and is in regeneration mode. Adsorbent with diminished capacity as compared to fresh or regenerated adsorbent may be termed spent adsorbent. It is not necessary for the adsorbent to reach zero capacity, and regeneration may be performed at any point of diminished adsorbent capacity. It is advantageous to employ tail gas stream 136 from bioreactor 100 in the gas fermentation zone for adsorbent regeneration. The bioreactor(s) of gas fermentation zone generate tail gas stream 136 and target product stream 138. Tail gas stream 136 passes through valve 140 and regeneration heater 142 to heat tail gas stream 136 and generate heated tail gas stream 144. Heated tail gas stream 144 passes through open valve 126, while valves 128 and 124 are closed, and to second adsorbent bed 118 providing heated regeneration gas to desorb contaminant on the adsorbent of second adsorbent bed 118. Additionally, valve 112 and valve 132 are closed and valve 150b is open.

The regeneration temperature is higher than the adsorption temperature. In some embodiments, the regeneration temperature is in the range from about 150° C. to about 200° C. In various embodiments, the regeneration temperature may be for example, from about 150° C. to 160° C., or from about 160° C. to about 180° C., or from about 180° C. to about 200° C.

The effluent stream 146b generated during regeneration of the second adsorbent bed 118 exits from TSA bed 118 and is passed to a thermal oxidizer or other energy recovery or oxidation device 400. Valve 322 is used to control the flow of effluent stream 146a r. Desorbed compounds from the TSA beds contain trace impurities e.g., volatile organic compounds enriched relative to the feed gas. The thermal oxidizer thermally destroys the trace impurities and generally recovers waste heat to enhance energy of the regeneration gas i.e., the tail gas stream 160. When TSA bed 118 is fully regenerated and operational, valve 112 and valve 132 are opened and valve 126 and valve 150b are closed.

Using heated tail gas stream 144 for regeneration of the second adsorbent bed 118 eliminates requirement of using inert gas e.g., nitrogen for TSA regeneration. Techniques for utilizing nitrogen require production and storage of nitrogen at a capital expenditure, followed by a large volume of contaminated nitrogen which may be treated, along with a dilute tail gas stream that has low energy value. Also, regeneration of the adsorbent beds by the tail gas stream from the bioreactor is advantageous over regeneration by product gas, as generally up to about 10% by volume of feed gas is lost during regeneration because the desorbed compounds contained therein are difficult to recycle without further treatment/separation.

Regeneration heater 142, in some embodiments is a fired heater providing direct heat transfer from the combustion of fuels. The heat energy is released by combusting fuels into an open space and transferred to the gas/fluids inside tubes which are arranged along the walls and roofs of the combustion chamber. In another embodiment, the regeneration heater includes an electric fired heater which uses electricity to increase the temperature of the tail gas stream. Depending on the application, electric fired heaters may be used for both direct and indirect heating. The electric fired heaters may include, but not limited to, immersion heater, circulation heater and electric thermal fluid heater. In another embodiment, saturated or superheated steam be used for heating. Other heat sources of suitable temperature can be envisioned within these embodiments.

In order to cool second adsorption bed 118 after it has been regenerated, treated gaseous feedstock from the first absorbent bed 116 is in fluid communication with the second adsorbent bed 118. A portion of the treated gaseous feedstock 122a from the first TSA bed 116 passes through second TSA bed 118 by opening valve 132 and valve 150b producing the effluent stream 146b which joins the regeneration effluent gas header 410. At least a portion of the regeneration effluent gas 410 r passes through a desorption cooler 240 by opening valve 318 to produce a cooling stream 192 which is compressed by a compressor 412 to overcome the pressure drop of the second TSA bed 118 and associated piping and valves. The compressed gas 192 is then combined into the product gas header 122 by opening valve 326 enabling recovery of the product gas used for cooling the second adsorbent bed 118, as illustrated in FIG. 1A. Using treated gaseous feedstock from the TSA bed for cooling and further recycling to the feed gas aids achieving nearly 100% recovery of the feed gas. FIGS. 1A and 1B show the cooling stream conduits as integrated with the regeneration stream conduits, but in other embodiments the conduits for each function may be independent.

In another embodiment, as illustrated in FIG. 1B, the cooling regeneration gas is recycled through the second TSA bed 118 by opening valve 226, valve 250b, valve 218, and valve 426 with valve 124, valve 128, valve 150a, valve 150c, valve 322 being closed. Once the adsorbent in the second TSA bed 118 approaches the adsorption temperature, the gas recirculation is stopped and the remaining gas in the second TSA bed 118 is recovered in the product gas header once the second TSA bed 118 is transitioned to the adsorption phase.

The thermal oxidizer 400 heats the trace impurities such as volatile organic compounds (VOC's) to a certain temperature, typically above the autoignition temperature, until they are oxidized. The oxidation process breaks down the harmful particulates into carbon dioxide, water, and trace quantities of other combustion byproducts. The thermal oxidizer may be selected from a direct fired thermal oxidizer, a recuperative thermal oxidizer, a regenerative thermal oxidizer, a catalytic thermal oxidizer, a flameless thermal oxidizer, or any combination thereof. The first type of thermal oxidizer is direct-fired thermal oxidizer. The direct fixed oxidizer operates on the principle that the combustion process gas stream is brought into a section of the direct thermal oxidizer, in which the temperature of the process gas stream is raised at or above the autoignition temperature and held in the furnace section at this temperature for a required residence time in order to achieve the desired VOC destruction efficiency. The second type of thermal oxidizer is regenerative thermal oxidizer (RTO). RTOS use a ceramic bed which may be heated from a previous oxidation cycle to preheat the input gases to partially oxidize them. The preheated gases enter a combustion chamber that may be heated by an external fuel source to reach the target oxidation temperature. This type of oxidizer is specifically designed for oxidizing large process gas streams having low organic compound concentrations, such as low percentages of organic pollutants including VOCs in the process gas stream. The third type of thermal oxidizer is a recuperative thermal oxidizer. Recuperative thermal oxidizers have a primary and/or secondary heat exchanger within the device. A primary heat exchanger preheats an incoming combustion air stream by recuperating heat from an existing clean gas stream. This primary heat recovery raises the temperature of the process gas stream before entering the combustion chamber, resulting in lower fuel requirements for the oxidizer device.

According to above discussion, the process for integrating TSA process and gas fermentation process includes steps of (a) providing a first TSA bed comprising an adsorbent and a second TSA bed comprising a spent adsorbent and a gas fermentation process comprising a bioreactor (b) passing a gaseous feedstock and at least one contaminant to the first TSA bed and generating a treated gaseous feedstock depleted in the contaminant (c) passing a portion of the treated gaseous feedstock to the gas fermentation process to generate a tail gas stream and a gas fermentation product stream (d) first passing at least a portion of the tail gas stream from the gas fermentation process through a heater to heat the tail gas stream to the required regeneration temperature and then pass the heated tail gas stream to the second TSA bed to desorb the adsorbed contaminant and provide a heated regenerated adsorbent (c) then passing at least a portion of the treated gaseous feedstock from the first TSA bed to the second TSA bed and through the regenerated adsorbent to cool the heated regenerated adsorbent to the adsorption temperature and provide a regeneration effluent stream (f) cooling and recompressing the regeneration effluent stream and combining the regeneration effluent stream from the second TSA bed with the treated gaseous feedstock from the first TSA bed or passing the effluent stream of the treated gaseous feedstock to the gas fermentation process, recycling the cooled regeneration effluent stream through the second TSA bed 118 to further cool the regenerated adsorbent, or any combination thereof. When the steps (a) to (f) are repeated, switching the adsorption switches from the first adsorption bed to the second adsorption bed and vice-versa periodically, in which one of the TSA bed comprises the adsorbent and the other TSA bed comprises the spent adsorbent. In another embodiment, a third TSA bed 120 comprising regenerated adsorbent is maintained at standby mode. The third TSA bed can be switched in operation if both the first TSA bed 116 and the second TSA bed 118 need to be offline because the adsorbents contained therein can no longer adsorb contaminant or to support extended regeneration cycles or maintenance requirements. In such circumstances, the third TSA bed 120 will be in fluid communication with the bioreactor 100. The third TSA bed 120 will be used for regeneration of either the first TSA bed 116 or the second TSA bed 118. The third TSA bed 120 can receive gaseous feedstock when the valve 114 is opened.

In one embodiment, the first TSA bed 116 may be pressurized to correspond to the pressure of the gas fermentation feedstock so that the adsorption occurs at the respective pressure. The pressure of the second TSA bed 118 may be lowered (depressurized) to correspond with the pressure of the tail gas stream 136 or the pressure of the heated tail gas stream 144 from the gas fermentation process so that regeneration occurs at respective pressure.

In some embodiments, the tail gas stream 136, or heated tail gas stream 144, from the bioreactor 100 passes through a scrubber 146. The scrubber advantageously removes traces of product stream such as ethanol in the tail gas stream 136 or heated tail gas stream 144. Therefore, passing the tail gas stream 136 or heated tail gas stream 144 through the scrubber 146 avoids additional cleaning of the TSA bed adsorption beds and prevents loss of the product produced by the bioreactor 100 that might be included in tail gas stream 136.

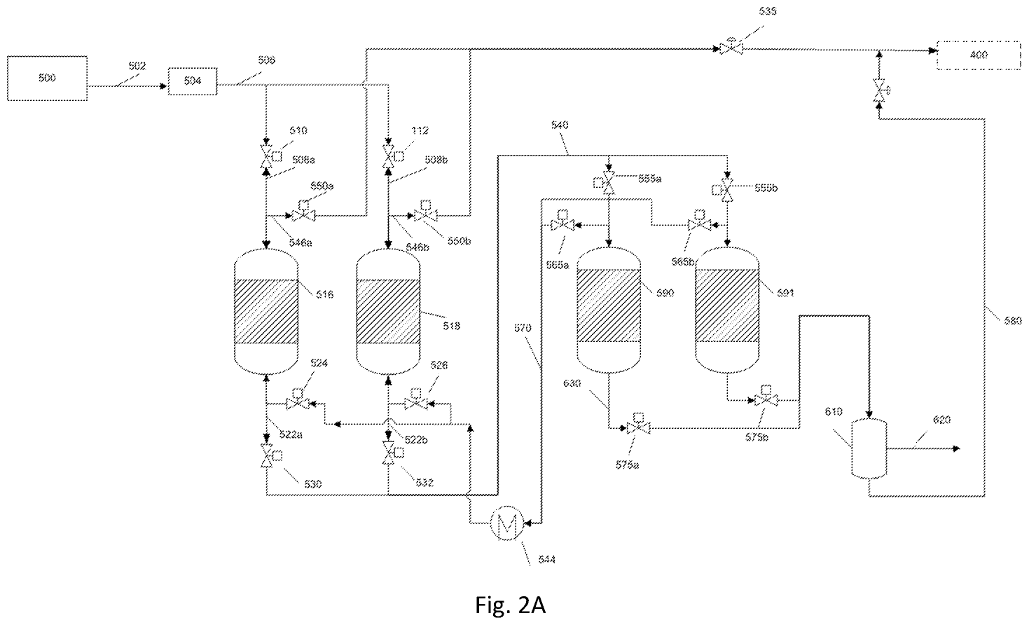

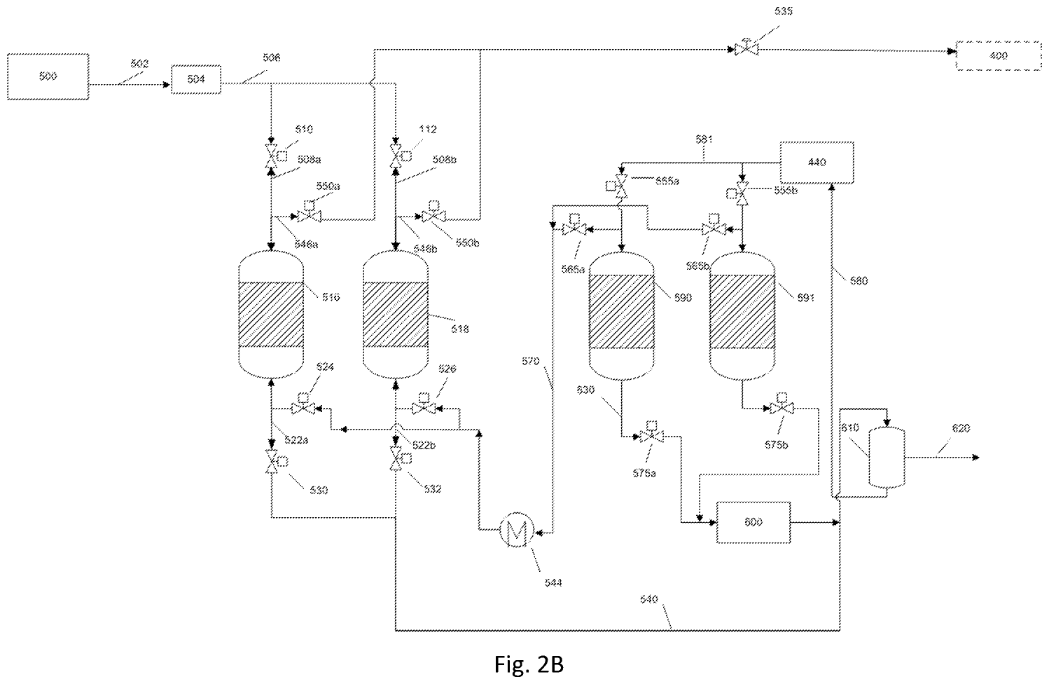

Referring to FIGS. 2A and 2B, in accordance with an alternative embodiment, an integrated TSA bed and PSA bed is disclosed. TSA is discussed above. Pressure swing adsorption (PSA) uses beds of solid adsorbent to separate components such as contaminants. The beds are then regenerated by depressurizing. The PSA technology is based on affinity of gas molecules to reversibly bind to the adsorbent material. The respective force acting between the gas molecules and the adsorbent material depends on the gas component, type of the adsorbent material, partial pressure of the gas component, and operating temperature. The separation effect is based on differences in binding forces to the adsorbent material. The PSA process works generally at constant temperature and uses the effect of alternating pressure and species partial pressure to perform adsorption and desorption. Since heating or cooling is not required, short cycles may be achieved. The process consequently allows the economical removal of large amounts of impurities. Adsorption is carried out at high pressure (and hence high respective partial pressure) typically in the range from about 10 bar to about 40 bar until the equilibrium loading is reached. At this point in time, no further adsorption capacity is available, and the adsorbent material may be regenerated. Such regeneration is accomplished by adjusting the pressure to slightly above or below atmospheric pressure resulting in a respective decrease in equilibrium loading. As a result, the contaminants on the adsorbent material are desorbed, and the adsorbent material is regenerated. After termination of the regeneration, pressure is increased back to adsorption pressure level, and the process starts again from the beginning.

As shown in FIG. 2A, the integrated TSA, the PSA and the gas fermentation process and device comprises the first TSA bed 516 comprising the adsorbent, the second TSA bed 518 comprising the spent adsorbent i.e., the adsorbent which can no longer sufficiently adsorb contaminant, a first PSA bed 590 comprising the PSA adsorbent, a second PSA bed 591 comprising the PSA spent adsorbent and a gas fermentation process in a bioreactor 610. Source 500 of gaseous feedstock provides gaseous feedstock stream 502. Gaseous feedstock 502 is optionally compressed in a compressor 504 to generate compressed gaseous feedstock 506. Compressed gaseous feedstock 506 enters first TSA bed 516 when the feedstock inlet valve 510 is opened to allow the gaseous feedstock 506 to enter into the first TSA bed 516 through inlet stream 508a for adsorption. The first TSA bed 516 operates at an adsorption temperature as described above for example and adsorbs at least one contaminant from the gaseous feedstock to generate a TSA treated gaseous feedstock 522a depleted in contaminant. However, the TSA treated gaseous feedstock may contain excess CO₂ or other components that may be removed. First PSA bed 590 is in fluid communication with the first TSA bed such that the TSA treated gaseous feedstock 522a depleted in a contaminant enters the first PSA bed 590 through stream 540 and passes through an adsorbent for adsorbing CO₂ or other components in first PSA bed 590 to produce a first PSA bed treated gaseous feedstock 630 enriched in fermentation substrate species such as CO and H₂. As used herein, the term “enriched” means that the outlet stream has a greater concentration of the indicated component than in the inlet stream to a vessel. The first PSA bed 590 is in fluid communication with the bioreactor 610 such that the first PSA bed treated gaseous feedstock 630 enters the bioreactor 610 through valve 575a. The bioreactor 610 generates a gas fermentation product stream 600 comprising for example, ethanol. CO₂ or other component removal by the first PSA bed 590 upfront before the gas fermentation aids to enrich the feedstock for the bioreactor 610 in feedstock components advantageous to certain gas fermentations and may enhance yield of product in the product stream 620. The spent adsorbent in second PSA bed 591 is regenerated by lowering the operating pressure forming a PSA reject stream 570 which can be used as regeneration gas for the TSA unit. The PSA reject stream is heated up to the TSA regeneration heater 544 using suitable heat sources as described previously. The heated PSA reject gas is then passed through the spent adsorbent in second TSA bed 518 transferring heat to the spent adsorbent. As the adsorbent temperature increases, the adsorbed contaminants are desorbed and carried out of the TSA bed by the regeneration gas forming a regeneration effluent stream 546b which is sent to a thermal oxidizer or other suitable process. Following adsorbent heating, the heater 544 duty is required and ultimately turned off allowing the cool PSA reject stream to cool the adsorbent down to or near the adsorption temperature as illustrated in FIG. 2A.

In another embodiment, a TSA unit is utilized to remove contaminants in the feed gas and a PSA unit is used to remove contaminants from the fermentation tail gas stream, as illustrated in FIG. 2B. In this configuration, fermentation tail gas stream 580 is optionally sent to scrubber 440 to recover trace quantities of fermentation product contained in fermentation tail gas stream 580. Scrubbed tail gas stream 581 is then routed to a PSA unit to remove bulk contaminants such as carbon dioxide to enrichen the concentration of gas fermentation substrates such as CO and H₂ before sending the gas to bioreactor 610. The PSA reject stream 570 is used as a regeneration gas for the second TSA bed as outlined above.

Recitation of ranges of values herein are merely intended to serve as a shorthand method of referring individually to each separate value falling within the range, unless otherwise indicated herein, and each separate value is incorporated into the specification as if it were individually recited herein. For example, unless otherwise indicated, any concentration range, percentage range, ratio range, integer range, size range, or thickness range is to be understood to include the value of any integer within the recited range and, when appropriate, fractions thereof (such as one tenth and one hundredth of an integer).

All methods described herein can be performed in any suitable order unless otherwise indicated herein or otherwise clearly contradicted by context. The use of any and all examples, or exemplary language (i.e., “such as”) provided herein, is intended merely to better illuminate the disclosure, and does not pose a limitation on the scope of the disclosure unless otherwise claimed. No language in the specification should be construed as indicating any non-claimed element as essential to the practice of the disclosure.

### EMBODIMENTS OF THE DISCLOSURE

Embodiments of this disclosure are described herein. Variations of those embodiments may become apparent to those of ordinary skill in the art upon reading the foregoing description, and employment of such variations as appropriate, is intended to be within the scope as the disclosure may be practiced otherwise than as specifically described herein. Accordingly, this disclosure includes all modifications and equivalents of the subject matter recited in the claims as permitted by applicable law. Moreover, any combination of the above described elements in all possible variations thereof is encompassed by the disclosure unless otherwise indicated herein or otherwise clearly contradicted by context.

Embodiment 1. A process for an integrated temperature swing adsorption process and gas fermentation process comprising:
(a) providing a temperature swing adsorption process comprising at least a first adsorption bed comprising adsorbent and a second adsorption bed comprising spent adsorbent and a gas fermentation process comprising a bioreactor comprising at least one C1-fixing microorganism in a nutrient solution;
(b) passing a gaseous feedstock comprising CO, CO₂, H₂, CH₄, or any combination thereof, and at least one contaminant to the first adsorption bed operating at an adsorption temperature, adsorbing the contaminant on the adsorbent, and generating a treated gaseous feedstock depleted in the contaminant;
(c) passing at least a portion of the treated gaseous feedstock to the gas fermentation process to generate a heated tail gas stream and a gas fermentation product stream comprising a gas fermentation product;
(d) first passing at least a portion of the heated tail gas stream from the gas fermentation process at a regeneration temperature to the second adsorption bed and through the spent adsorbent to desorb the adsorbed contaminant and provide a heated regenerated adsorbent, wherein the regeneration temperature is greater than the adsorption temperature;
(c) then passing at least a portion of the treated gaseous feedstock from the first adsorption bed to the second adsorption bed and through the regenerated adsorbent to cool the heated regenerated adsorbent to the adsorption temperature and provide an effluent stream of the treated gaseous feedstock; and
(f) combining the effluent stream of treated gaseous feedstock from the second adsorption bed with the treated gaseous feedstock from the first adsorption bed or passing the effluent stream of the treated gaseous feedstock to the gas fermentation process, or both.

Embodiment 2. The process of embodiment 1, further comprising periodically repeating the process.

Embodiment 3. The process of embodiment 1 or 2, further comprising a third adsorption bed comprising regenerated adsorbent in standby mode.

Embodiment 4. The process of any of the preceding embodiments, further comprising passing the hot heated tail gas stream from the gas fermentation process through a scrubber prior to passing to the first adsorption bed to remove and recover gas fermentation product from the heated tail gas stream.

Embodiment 5. The process of any of the preceding embodiments, wherein the adsorption temperature is in the range of from about 40° C. to about 60° C.

Embodiment 6. The process of any of the preceding embodiments, wherein the regeneration temperature is in the range of from about 150° C. to about 200° C.

Embodiment 7. The process of any of the preceding embodiments, wherein the gaseous feedstock is selected from an industrial waste gas, a syngas, a biogas, a landfill gas, a direct air capture, or any combination thereof.

Embodiment 8. The process of any of the preceding embodiments, wherein the syngas is generated by a reforming process, a partial oxidation process, a gasification process, or any combination thereof.

Embodiment 9. The process of any of the preceding embodiments, wherein the industrial gas is obtained from a source selected from steel manufacturing, non-ferrous products manufacturing, petroleum refining, electric power production, carbon black production, paper and pulp manufacturing, ammonia production, methanol production, coke manufacturing, petrochemical production, carbohydrate fermentation, cement making, aerobic digestion, anaerobic digestion, natural gas extraction, cellulosic fermentation, oil extraction, industrial processing of geological reservoirs, processing fossil resources, or any combination thereof.

Embodiment 10. The process of any of the preceding embodiments, wherein the contaminant is selected from at least one of a hydrocarbon, an oxygenate, a sulfur compound, a nitrogen compound, or any combination thereof.

Embodiment 11. The process of any of the preceding embodiments, wherein the adsorbent or the spent adsorbent is selected from zeolitic molecular sieves, activated carbon, silica gel, activated aluminas, or any combination thereof.

Embodiment 12. The process of any of the preceding embodiments, further comprising adjusting the operating pressure of: (a) the first adsorption bed to correspond with the pressure of the gaseous feedstock; (b) the second adsorption bed to correspond with the pressure of the heated tail gas stream from the gas fermentation process; or both (a) and (b).

Embodiment 13. A process for an integrated temperature swing adsorption process, pressure swing adsorption process, and gas fermentation process comprising:
providing a temperature swing adsorption process comprising at least a first TSA adsorption bed comprising TSA adsorbent and a second TSA adsorption bed comprising TSA spent adsorbent, a pressure swing adsorption process comprising at least a first PSA adsorption bed comprising PSA adsorbent and a second PSA adsorption bed comprising PSA spent adsorbent, and a gas fermentation process comprising a bioreactor comprising at least one C1-fixing microorganism in a nutrient solution; passing a gaseous feedstock comprising CO, CO₂, H₂, CH₄, or any combination thereof, and at least one contaminant to the first TSA adsorption bed operating at an adsorption temperature, adsorbing the contaminant on the TSA adsorbent, and generating a TSA treated gaseous feedstock depleted in the contaminant; passing at least a portion of the treated gaseous feedstock to the first PSA adsorption bed operating at an adsorption pressure, adsorbing CO₂ on the PSA adsorbent, and generating a PSA treated gaseous feedstock depleted in CO₂; passing the PSA treated gaseous feedstock to a gas fermentation process to generate a gas fermentation product stream comprising a gas fermentation product; first passing at least a portion of the TSA treated gaseous feedstock to the second PSA adsorption bed operating at a desorption pressure, desorbing CO₂ from the PSA spent adsorbent, and generating a PSA purge gas stream enriched in CO, wherein the desorption pressure is different from the adsorption pressure; and then passing at least a portion of the PSA purge gas stream from the second PSA adsorption bed to the second TSA adsorption bed at a desorption temperature to desorb the adsorbed contaminant and provide a TSA purge gas comprising the contaminant and CO₂.

Embodiment 14. A process for an integrated temperature swing adsorption process, pressure swing adsorption process, and gas fermentation process comprising:
providing a temperature swing adsorption process comprising at least a first TSA adsorption bed comprising TSA adsorbent and a second TSA adsorption bed comprising TSA spent adsorbent, a pressure swing adsorption process comprising at least a first PSA adsorption bed comprising PSA adsorbent and a second PSA adsorption bed comprising PSA spent adsorbent, and a gas fermentation process comprising a bioreactor comprising at least one C1-fixing microorganism in a nutrient solution;
passing a gaseous feedstock comprising CO, CO₂, H₂, CH₄, or any combination thereof, and at least one contaminant to the first TSA adsorption bed operating at an adsorption temperature, adsorbing the contaminant on the TSA adsorbent, and generating a TSA treated gaseous feedstock depleted in the contaminant;
passing at least a portion of the treated gaseous feedstock to a gas fermentation process to generate a fermentation tail gas and a gas fermentation product stream comprising a gas fermentation product;
passing at least a portion of the fermentation tail gas to the first PSA adsorption bed operating at an adsorption pressure, adsorbing CO₂ on the PSA adsorbent, and generating a PSA treated fermentation tail gas depleted in CO₂ and PSA purge gas;
passing at least a portion of the PSA purge gas from the first PSA adsorption bed to the second TSA adsorption bed at a desorption temperature to desorb the adsorbed contaminant and provide a TSA purge gas comprising the contaminant and CO₂.

Embodiment 15. An integrated temperature swing adsorption and gas fermentation device comprising:
a gaseous feedstock source unit in fluid communication with a first adsorption bed comprising an adsorbent, the first adsorption bed further comprising a treated gaseous feedstock outlet;
a second adsorption bed comprising a spent adsorbent and having an effluent outlet wherein the treated gaseous feedstock outlet of the first adsorption bed is in further fluid communication with the second adsorption bed; and
a bioreactor of a gas fermentation device in fluid communication with the treated gaseous feedstock outlet of the first adsorption bed and the effluent outlet of the second adsorption bed, the bioreactor comprising a tail gas stream outlet and a product stream outlet, the tail gas stream outlet in fluid communication with the second adsorption bed.

Embodiment 16. The device of embodiment 15, further comprising a third adsorption bed comprising regenerated adsorbent in standby mode, the third adsorption bed is in fluid communication with the bioreactor.

Embodiment 17. The device of embodiment 15, wherein a scrubber is in fluid communication with the tail gas stream outlet of the bioreactor, the scrubber is in further fluid communication with the second adsorption bed.

Embodiment 18. The device of any of embodiments 15, wherein each of the temperature swing adsorption bed and the bioreactor is in fluid communication with at least one on-off valve.

Embodiment 19. The device of any of embodiments 15, further comprising a regeneration heater in fluid communication with the tail gas stream outlet of the bioreactor.

Embodiment 20. An integrated temperature swing adsorption, pressure swing adsorption and gas fermentation device comprising:
a gaseous feedstock source unit in fluid communication with a first TSA adsorption bed comprising an adsorbent, the first TSA adsorption bed further comprising a treated gaseous feedstock outlet;
a first PSA adsorption bed comprising PSA adsorbent, the first PSA bed in fluid communication with the treated gaseous feedstock outlet of the first TSA adsorption bed wherein the first PSA adsorption bed is in further fluid communication with the bioreactor; and a second PSA adsorption bed comprising PSA spent adsorbent in fluid communication with the treated gaseous feedstock outlet of the first TSA adsorption bed wherein the second PSA adsorption bed is in further fluid communication with a second TSA adsorption bed comprising TSA spent adsorbent.

1. A process for an integrated temperature swing adsorption process and gas fermentation process comprising:
(a) providing a temperature swing adsorption process comprising at least a first adsorption bed comprising adsorbent and a second adsorption bed comprising spent adsorbent and a gas fermentation process comprising a bioreactor comprising at least one C1-fixing microorganism in a nutrient solution;
(b) passing a gaseous feedstock comprising CO, CO₂, H₂, CH₄, or any combination thereof, and at least one contaminant to the first adsorption bed operating at an adsorption temperature, adsorbing the contaminant on the adsorbent, and generating a treated gaseous feedstock depleted in the contaminant;
(c) passing at least a portion of the treated gaseous feedstock to the gas fermentation process to generate a heated tail gas stream and a gas fermentation product stream comprising a gas fermentation product;
(d) first passing at least a portion of the heated tail gas stream from the gas fermentation process at a regeneration temperature to the second adsorption bed and through the spent adsorbent to desorb the adsorbed contaminant and provide a heated regenerated adsorbent, wherein the regeneration temperature is greater than the adsorption temperature;
(e) then passing at least a portion of the treated gaseous feedstock from the first adsorption bed to the second adsorption bed and through the regenerated adsorbent to cool the heated regenerated adsorbent to the adsorption temperature and provide an effluent stream of the treated gaseous feedstock; and
(f) combining the effluent stream of treated gaseous feedstock from the second adsorption bed with the treated gaseous feedstock from the first adsorption bed or passing the effluent stream of the treated gaseous feedstock to the gas fermentation process, or both.

2. The process of claim 1, further comprising periodically repeating the process.

3. The process of claim 1, further comprising a third adsorption bed comprising regenerated adsorbent in standby mode.

4. The process of claim 1, further comprising passing the heated tail gas stream from the gas fermentation process through a scrubber prior to passing to the first adsorption bed to remove and recover gas fermentation product from the heated tail gas stream.

5. The process of claim 1, wherein the adsorption temperature is in the range of from about 40° C. to about 60° C.

6. The process of claim 1, wherein the regeneration temperature is in the range of from about 150° C. to about 200° C.

7. The process of claim 1, wherein the gaseous feedstock is selected from an industrial waste gas, a syngas, a biogas, a landfill gas, a direct air capture, or any combination thereof.

8. The process of claim 1, wherein the syngas is generated by a reforming process, a partial oxidation process, a gasification process, or any combination thereof.

9. The process of claim 1, wherein the industrial gas is obtained from a source selected from steel manufacturing, non-ferrous products manufacturing, petroleum refining, electric power production, carbon black production, paper and pulp manufacturing, ammonia production, methanol production, coke manufacturing, petrochemical production, carbohydrate fermentation, cement making, aerobic digestion, anaerobic digestion, natural gas extraction, cellulosic fermentation, oil extraction, industrial processing of geological reservoirs, processing fossil resources, or any combination thereof.

10. The process of claim 1, wherein the contaminant is selected from at least one of a hydrocarbon, an oxygenate, a sulfur compound, a nitrogen compound, or any combination thereof.

11. The process of claim 1, wherein the adsorbent or the spent adsorbent is selected from zeolitic molecular sieves, activated carbon, silica gel, activated aluminas, or any combination thereof.

12. The process of claim 1, further comprising adjusting the operating pressure of:
(a) the first adsorption bed to correspond with the pressure of the gaseous feedstock;
(b) the second adsorption bed to correspond with the pressure of the heated tail gas stream from the gas fermentation process; or
(c) both (a) and (b).

13. A process for an integrated temperature swing adsorption process, pressure swing adsorption process, and gas fermentation process comprising:
(a) providing a temperature swing adsorption process comprising at least a first TSA adsorption bed comprising TSA adsorbent and a second TSA adsorption bed comprising TSA spent adsorbent, a pressure swing adsorption process comprising at least a first PSA adsorption bed comprising PSA adsorbent and a second PSA adsorption bed comprising PSA spent adsorbent, and a gas fermentation process comprising a bioreactor comprising at least one C1-fixing microorganism in a nutrient solution;
(b) passing a gaseous feedstock comprising CO, CO₂, H₂, CH₄, or any combination thereof, and at least one contaminant to the first TSA adsorption bed operating at an adsorption temperature, adsorbing the contaminant on the TSA adsorbent, and generating a TSA treated gaseous feedstock depleted in the contaminant;
(c) passing at least a portion of the treated gaseous feedstock to the first PSA adsorption bed operating at an adsorption pressure, adsorbing CO₂ on the PSA adsorbent, and generating a PSA treated gaseous feedstock depleted in CO₂;
(d) passing the PSA treated gaseous feedstock to a gas fermentation process to generate a gas fermentation product stream comprising a gas fermentation product;
(e) first passing at least a portion of the TSA treated gaseous feedstock to the second PSA adsorption bed operating at a desorption pressure, desorbing CO₂ from the PSA spent adsorbent, and generating a PSA purge gas stream enriched in CO, wherein the desorption pressure is different from the adsorption pressure; and
(f) then passing at least a portion of the PSA purge gas stream from the second PSA adsorption bed to the second TSA adsorption bed at a desorption temperature to desorb the adsorbed contaminant and provide a TSA purge gas comprising the contaminant and CO₂.

14. A process for an integrated temperature swing adsorption process, pressure swing adsorption process, and gas fermentation process comprising:
(a) providing a temperature swing adsorption process comprising at least a first TSA adsorption bed comprising TSA adsorbent and a second TSA adsorption bed comprising TSA spent adsorbent, a pressure swing adsorption process comprising at least a first PSA adsorption bed comprising PSA adsorbent and a second PSA adsorption bed comprising PSA spent adsorbent, and a gas fermentation process comprising a bioreactor comprising at least one C1-fixing microorganism in a nutrient solution;
(b) passing a gaseous feedstock comprising CO, CO₂, H₂, CH₄, or any combination thereof, and at least one contaminant to the first TSA adsorption bed operating at an adsorption temperature, adsorbing the contaminant on the TSA adsorbent, and generating a TSA treated gaseous feedstock depleted in the contaminant;
(c) passing at least a portion of the treated gaseous feedstock to a gas fermentation process to generate a fermentation tail gas and a gas fermentation product stream comprising a gas fermentation product;
(d) passing at least a portion of the fermentation tail gas to the first PSA adsorption bed operating at an adsorption pressure, adsorbing CO₂ on the PSA adsorbent, and generating a PSA treated fermentation tail gas depleted in CO₂ and PSA purge gas;
(e) passing at least a portion of the PSA purge gas from the first PSA adsorption bed to the second TSA adsorption bed at a desorption temperature to desorb the adsorbed contaminant and provide a TSA purge gas comprising the contaminant and CO₂.

15. An integrated temperature swing adsorption and gas fermentation device comprising:
i. a gaseous feedstock source unit in fluid communication with a first adsorption bed comprising an adsorbent, the first adsorption bed further comprising a treated gaseous feedstock outlet;
ii. a second adsorption bed comprising a spent adsorbent and having an effluent outlet wherein the treated gaseous feedstock outlet of the first adsorption bed is in further fluid communication with the second adsorption bed; and
iii. a bioreactor of a gas fermentation device in fluid communication with the treated gaseous feedstock outlet of the first adsorption bed and the effluent outlet of the second adsorption bed, the bioreactor comprising a tail gas stream outlet and a product stream outlet, the tail gas stream outlet in fluid communication with the second adsorption bed.

16. The device of claim 15, further comprising a third adsorption bed comprising regenerated adsorbent in standby mode, the third adsorption bed is in fluid communication with the bioreactor.

17. The device of claim 15, wherein a scrubber is in fluid communication with the tail gas stream outlet of the bioreactor, the scrubber is in further fluid communication with the second adsorption bed.

18. The device of claim 15, wherein each of the temperature swing adsorption bed and the bioreactor is in fluid communication with at least one on-off valve.

19. The device of claim 15, further comprising a regeneration heater in fluid communication with the tail gas stream outlet of the bioreactor.
